(19) United States
(12) Patent Application Publication
(10) Pub. No.: US 2003/0105396 A1
(43) Pub. Date: Jun. 5, 2003

(54) Diagnostic imaging

(76) Inventors: Morten Eriksen, Oslo (NO); Jonny Ostensen, Oslo (NO)

Correspondence Address: Amersham Biosciences Corp, 800 Centennial Avenue, Piscataway, NJ, 08855, US

(21) Appl. No.: 10/302,691

(22) Filed: Nov. 22, 2002

Related U.S. Application Data
(63) Continuation of application No. PCT/NO01/00227, filed on Jun. 1, 2001.
(60) Provisional application No. 60/211,336, filed on Jun. 13, 2000.

(30) Foreign Application Priority Data
Jun. 1, 2000 (GB) 0013352.0

(51) Int. Cl. A61B 6/00

(52) U.S. Cl. 600/431; 600/421

(57) ABSTRACT

The invention relates to the administration of dynamic, particulate dispersion systems, e.g. gas-containing diagnostic contrast agents, more particularly to apparatus and a method for the controlled and substantially steady state administration of such gravity segregating dispersions by infusion. Controlled delivery of substantially homogeneous gravity segregating dispersion, e.g. gas-containing contrast agent, may be achieved by an infusion procedure in which the dispersion is delivered from a syringe or other preferably cylinder shaped reservoir, e.g. tubing, which is exposed to a thermal gradient across its body.

This invention relates to the administration of dynamic, particulate dispersion systems, e.g. gas-containing diagnostic contrast agents, more particularly to apparatus and a method for the controlled and substantially steady state administration of such gravity segregating dispersions by infusion.

In the field of ultrasonography it is well known that contrast agents comprising dispersions of gas microbubbles are particularly efficient backscatterers of ultrasound by virtue of the low density and ease of compressibility of the microbubbles. Such microbubble dispersions, if appropriately stabilised, may permit highly effective ultrasound visualisation of, for example, the vascular system and tissue microvasculature, often at advantageously low doses of the contrast agent.

Gas-containing contrast media are also known to be effective in magnetic resonance (MR) imaging, e.g. as susceptibility contrast agents which will act to reduce MR signal intensity. Oxygen-containing contrast media also represent potentially useful paramagnetic MR contrast agents.

In the field of X-ray imaging gases such as carbon dioxide may be used as intravascular contrast agents. Moreover, the use of radioactive gases, e.g. radioactive isotopes of inert gases such as xenon, has been proposed in scintigraphy, for example for blood pool imaging.

Gas-containing ultrasound contrast agents are usually administered intravenously as a single or multiple bolus dosage, leading to a rapid and pronounced but relatively short lived rise in backscatter intensity in respect of blood-perfused tissue and organs as the bolus mixes with surrounded blood and is carried through the circulation system. A plot of backscatter intensity against time therefore shows a relatively narrow and high signal intensity peak; backscatter measurements are normally made during the existence of this peak, although this may give rise to problems in, for example, the imaging of deeper tissue and organs where high backscatter from overlying tissue may cause excessive shadowing during the peak period.

As discussed in WO-A-9748337, diagnostic artefacts such as shadowing may be reduced by controlling the rate of administration of the contrast agent and/or by administering a flush such as normal saline after administration of the contrast agent. Contrast agent administration rates of 1-8×10⁶ vesicles/kg-sec or 1×10⁻⁷ to 3×10⁻³ cc gas/kg-sec and flush rates of 0.01-2.4 ml/sec are suggested; the contrast agent is typically administered over a period of 5-20 seconds, and any subsequent flush is typically administered over a period in the range 10 seconds to 10 minutes.

Continuous infusion of ultrasound contrast agents, for example over a period in the range from one minute to one hour, is of potential interest in that it may permit administration of the contrast agent at a rate which minimises diagnostic artefacts such as shadowing and may lengthen the useful time window for imaging beyond the relatively short duration of the backscatter signal peak resulting from passage of a contrast agent bolus.

Thus, for example, Albrecht et al. in Radiology 207, pp. 339-347 (1998) note that the use of continuous contrast agent infusion to provide prolonged enhancement of Doppler signals is advantageous in that it may permit completion of lengthy imaging procedures such as studies of the renal arteries or peripheral leg veins and may optimise dose effectiveness of the contrast agents, as well as reducing saturation artefacts.

Administration of contrast agents by infusion may also be useful in procedures based on imaging of contrast agent in the recirculating phase following admixture with the blood pool, as described in WO-A-9908714.

A problem with the continuous infusion of gas-containing diagnostic contrast agents arises from the tendency of the gas-filled components to float, since this will lead to inhomogeneities forming within vessels such as power-driven syringes which may be used to administer the contrast agent. This may, for example, lead to an increase in microbubble concentration in the upper part of such a vessel and/or to changes in size distribution occurring at various points within the vessel as larger microbubbles float more rapidly than smaller microbubbles.

A possible solution to this problem is proposed in WO-A-9927981, which discloses powered injector systems comprising a syringe which is subjected to rotational or rocking motion in order to maintain homogeneity within the contents thereof. In specific embodiments the barrel of the syringe is positioned horizontally in contact with wheels or moveable brackets which are capable of alternately rotating the syringe in opposite directions about is longitudinal (i.e. horizontal) axis.

It will be appreciated that the incorporation of such rotational or other agitational means into syringe driver apparatus necessarily complicates the design and significantly increases the cost of such apparatus, so that there is an ongoing need for apparatus which permits the continuous infusion of gravity segregating dispersions while maintaining substantial homogeneity of the dispersion.

The present invention is based on the finding that controlled delivery of substantially homogeneous gravity segregating dispersion, e.g. gas-containing contrast agent, may be achieved by an infusion procedure in which the dispersion is delivered from a syringe or other preferably cylinder shaped reservoir, e.g. tubing, which is exposed to a thermal gradient across its body.

By exposing one side of the reservoir to a higher temperature than the other side, a thermal imbalance is generated inside the reservoir by thermal conduction through its wall, and thus the local density of fluid within the reservoir is changed due to thermal expansion or contraction. The resulting density differential will cause motion of the fluid within the reservoir; such motion will have a stirring effect tending to maintain homogeneity of the dispersion.

This method does not require any expensive shaking apparatus and there is no requirement for any device, e.g. a stirrer bar, to be in direct contact with the fluid within the reservoir. Hence, the method eliminates the potential for damage to be caused to the dispersion, e.g. to microbubbles present in a contrast agent formulation, which a stirring device may cause. Moreover, once the thermal gradient has been established across the reservoir body, the method of the invention does not require the continuous attention of the practitioner. The fluid within the reservoir is a dispersion. The dispersion may by definition also be an emulsion or a suspension, and may comprise microbubbles that tend to float or particles that tend to sediment. A substantially homogeneous gravity segregating dispersion may be achieved by exposing the delivery reservoir to a thermal gradient. Preferably the dispersion is a formulation of microbubbles in a fluid. Thus, according to one aspect of the present invention there is provided a method of administering a gravity segregating dispersion, e.g. a gas-containing contrast agent, to a subject by continuous infusion, wherein said contrast agent is controllably delivered from a reservoir, e.g. a syringe, exposed to a thermal gradient across the body thereof.

According to a further aspect, the invention provides apparatus useful in the administration of a gravity segregating dispersion, e.g. a gas-containing contrast agent, by continuous infusion, said apparatus comprising (i) a reservoir adapted to retain a gravity segregating dispersion; (ii) means to induce a thermal gradient across the body of the reservoir; (iii) optionally means to allow co-administration of said gravity segregating dispersion with a flushing medium and (iv) conduit means for conducting said dispersion and optionally said flushing medium to an injection device.

A thermal gradient across the body of the reservoir may be created by any convenient means and may readily be achieved by the person skilled in the art. For example, the body of the reservoir may be exposed to heat from one side whilst the other side of the reservoir body is maintained at room temperature. Alternatively, one side of the reservoir body may be cooled and the other side maintained at room temperature. Preferably however, one side of the reservoir body is heated and the other side cooled. It is preferred if the average temperature of the heating and cooling sources is approximately equal to the ambient room temperature and hence simultaneous heating and cooling is most preferred.

Although it is not necessary for the heating or cooling sources (from hereon referred to as thermal sources) to contact the reservoir body, it is preferred that thermal contact does take place, since the resultant heating and/or cooling action is more efficient and controllable. For example, one side of the reservoir may be contacted with ice, cold water or cold air and the other side contacted with a heat source such as warm water, warm air or a warmed piece of metal.

Other ways of heating and cooling the reservoir, such as placing thermal sources near the reservoir, will be readily apparent to the skilled artisan.

It is essential to maintain the temperature within the reservoir at such a level such that no damage occurs to the dispersion therein. A heating source should therefore preferably not expose the reservoir contents to a temperature greater than 45ΕC. or less than 5ΕC. The temperatures of the actual thermal sources will of course vary depending on how close the thermal source is to the reservoir and how efficiently the source transfers its heat to or withdraws heat from the reservoir. When the thermal source is in contact with the reservoir it is preferred if the temperature of the thermal source does not exceed 50ΕC. or go below 0ΕC.

The reservoir could be any delivery vessel, but is preferably a syringe driver means comprising a syringe. To have an optimal stirring effect caused by the thermal gradient, the reservoir, and especially a syringe, should be shaped in such a way that recesses and corners are minimised. Especially this will be relevant for the ends of the chamber and the plunger of a syringe.

In a preferred embodiment, the thermal sources are solid and curved so as to contact more of the reservoir body surface. Preferably however, no one thermal source should contact more than half of the reservoir body and most preferably each thermal source should contact about one third of the reservoir surface. Also, it is preferred that the thermal sources contacting the reservoir be opposite each other in use. The thermal sources may be specifically crafted to ensure close contact with the reservoir body in which the gravity segregating dispersion is stored.

In a most preferred embodiment, the reservoir is held between two jaws, preferably of different temperatures, one of which is relatively hot, the other relatively cold. Although each jaw may come from a separate device, conveniently, both jaws are provided by a device such as a clamp optionally with Peltier element. The jaws may be made from a substance with high thermal conductivity, conveniently a metal such as aluminium, copper or silver or alloys of such metals. The jaws may also be made of steel, lead, brass, nickel, cadmium, tin or zink. The jaws may be heated or cooled by immersion in water of appropriate temperature. Alternatively, where the reservoir is held by a clamp with a Peltier element, combined heating and cooling may be achieved electrically. A typical Peltier element is supplied from a DC supply typically using 1.5 A at 6V requiring approximately 5 to 10 minutes to achieve the desired temperature gradient. The device may be prepared without a thermostat or other temperature control means and may be considered to be self-regulating. Since a Peltier element produces more thermal energy on the hot side than is removed from the cold side, the hot side of the assembly may advantageously be cooled by cooling fins.

It is preferred that the gravity segregating dispersion, e.g. gas-containing contrast agent is co-administration with a flushing medium to further enhance product homogeneity, e.g. by reducing residence time and thereby the effects of flotation in connecting tubing etc. By co-administering is meant that the dispersion is delivered from a delivery vessel, a reservoir, and thereafter is admixed with a flushing medium prior to administration to a subject. This co-administration also permits particularly efficient control of administration of the gas-containing contrast agent since the flow rates of both the gravity segregating dispersion and flushing medium may be independently controlled.

Co-administration also reduces the need for dilution of the contrast agent, this being favourable as contrast agents are often unstable when stored after dilution. Moreover, by co-administrating the gas-containing contrast agent with the flushing medium, the presence of an open injection route, independent of contrast agent flow and local blood flow variations is ensured.

The reservoir may be held at any convenient angle, e.g. horizontally or vertically. The optimal position of the reservoir is dependent on many factors, one important factor being how the thermal gradient is achieved.

By using an essentially vertically positioned reservoir the height of the contrast agent sample in the reservoir is greatly increased, thereby extending the distance through which flotation may occur. Since microbubbles of a given size will rise through carrier liquid at a constant rate, this significantly reduces the effects of flotation separation and thereby improves dose control over a given period of time.

The term “essentially vertical” as used herein denotes that the longitudinal axis of the reservoir, e.g. a syringe, should be positioned within about 30Ε of vertical, preferably within 15Ε and more preferably within 5Ε of vertical. The reservoir may be positioned for either upward or downward delivery of contrast agent. In the former case such flotation as occurs during administration of the contrast agent will tend to lead to a reduction in microbubble concentration as administration proceeds. Conversely the microbubble concentration will tend to increase during administration in the latter case. In either case this may, if desired, be counteracted by appropriate adjustment of the rate at which the contrast agent is administered with flushing medium. Also, it is envisaged that the essentially vertical reservoir, e.g. syringe, may be flipped at a suitable stage during infusion.

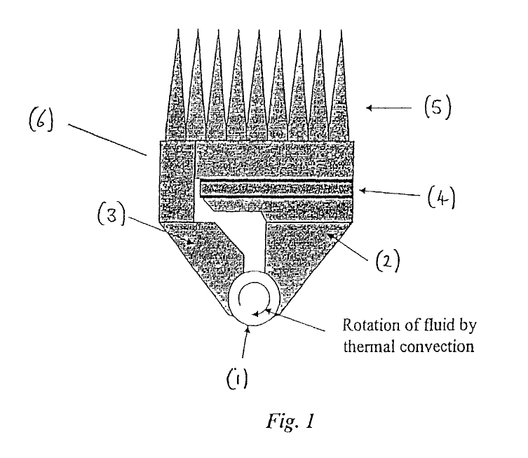

However, in a preferred embodiment, the reservoir is held essentially horizontally. In this way the thermal gradient applied across the body induces rotation of the dispersion within the reservoir by thermal convection as illustrated in FIG. 1.

The term “essentially horizontal” as used herein denotes that the longitudinal axis of the reservoir should be positioned within about 30Ε of horizontal, preferably within 15Ε and more preferably within 5Ε of horizontal.

Syringe driver means which may be used in accordance with the invention include power injection systems in which the syringe plunger is controllably driven by an appropriate automated mechanism, for example an electrically powered and controlled helical screw or push rod.

The rate at which the contrast agent is administered may, for example, be in the range 0.01-0.25 ml/minute, and may be selected to take account of factors such as the microbubble concentration and the desired degree of attenuation in ultrasound studies.

The flushing medium may be any appropriate biocompatible liquid, but is preferably normal saline. It may, for example, be administered by gravitational flow using appropriate flow rate controlling means, or may be delivered using a controllable pump. Flow rates of 1-2 ml/minute, have been found to be appropriate although higher flow rates may also be used.

Mixing of the contrast agent and flushing medium may, for example, be effected in a three way connector or tap which is also connected to an injection device such as a needle or catheter. It is preferred that connections are made using low volume tubing in order to minimise transit time of contrast agent and thus to minimise the potential for flotation separation of microbubbles.

Gases which may be present in contrast agents administered in accordance with the invention include any biocompatible substances, including mixtures, which are at least partially, e.g. substantially or completely, in gaseous or vapour form at the normal human body temperature of 37ΕC. Representative gases thus include air; nitrogen; oxygen; carbon dioxide; hydrogen; inert gases such as helium, argon, xenon or krypton; sulphur fluorides such as sulphur hexafluoride, disulphur decafluoride or trifluoromethylsulphur pentafluoride; selenium hexafluoride; optionally halogenated silanes such as methylsilane or dimethylsilane; low molecular weight hydrocarbons (e.g. containing up to 7 carbon atoms), for example alkanes such as methane, ethane, a propane, a butane or a pentane, cycloalkanes such as cyclopropane, cyclobutane or cyclopentane, alkenes such as ethylene, propene, propadiene or a butene, and alkynes such as acetylene or propyne; ethers such as dimethyl ether; ketones; esters; halogenated low molecular weight hydrocarbons (e.g. containing up to 7 carbon atoms); and mixtures of any of the foregoing. Advantageously at least some of the halogen atoms in halogenated gases are fluorine atoms; thus biocompatible halogenated hydrocarbon gases may, for example, be selected from bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethylfluoride, 1,1-difluoroethane and perfluorocarbons. Representative perfluorocarbons include perfluoroalkanes such as perfluoromethane, perfluoroethane, perfluoropropanes, perfluorobutanes (e.g. perfluoro-n-butane, optionally in admixture with other isomers such as perfluoro-iso-butane), perfluoropentanes, perfluorohexanes or perfluoroheptanes; perfluoroalkenes such as perfluoropropene, perfluorobutenes (e.g. perfluorobut-2-ene), perfluorobutadiene, perfluoropentenes (e.g. perfluoropent-1-ene) or perfluoro-4-methylpent-2-ene; perfluoroalkynes such as perfluorobut-2-yne; and perfluorocycloalkanes such as perfluorocyclobutane, perfluoromethylcyclobutane, perfluorodimethylcyclobutanes, perfluorotrimethyl-cyclobutanes, perfluorocyclopentane, perfluoromethyl-cyclopentane, perfluorodimethylcyclopentanes, perfluorocyclohexane, perfluoromethylcyclohexane or perfluorocycloheptane. Other halogenated gases include methyl chloride, fluorinated (e.g. perfluorinated) ketones such as perfluoroacetone and fluorinated (e.g. perfluorinated) ethers such as perfluorodiethyl ether. The use of perfluorinated gases, for example sulphur hexafluoride and perfluorocarbons such as perfluoropropane, perfluorobutanes, perfluoropentanes and perfluorohexanes, may be particularly advantageous in view of the recognised high stability in the blood stream of microbubbles containing such gases. Other gases with physicochemical characteristics which cause them to form highly stable microbubbles in the blood stream may likewise be useful.

Representative examples of contrast agent formulations include microbubbles of gas stabilised (e.g. at least partially encapsulated) by a coalescence-resistant surface membrane (for example gelatin, e.g. as described in WO-A-8002365), a filmogenic protein (for example an albumin such as human serum albumin, e.g. as described in U.S. Pat. No. 4,718,433, U.S. Pat. No. 4,774,958, U.S. Pat. No. 4,844,882, EP-A-0359246, WO-A-9112823, WO-A-9205806, WO-A-9217213, WO-A-9406477, WO-A-9501187 or WO-A-9638180), a polymer material (for example a synthetic biodegradable polymer as described in EP-A-0398935, an elastic interfacial synthetic polymer membrane as described in EP-A-0458745, a microparticulate biodegradable polyaldehyde as described in EP-A-0441468, a microparticulate N-dicarboxylic acid derivative of a polyamino acid—polycyclic imide as described in EP-A-0458079, or a biodegradable polymer as described in WO-A-9317718 or WO-A-9607434), a non-polymeric and non-polymerisable wall-forming material (for example as described in WO-A-9521631), or a surfactant (for example a polyoxyethylene-polyoxypropylene block copolymer surfactant such as a Pluronic, a polymer surfactant as described in WO-A-9506518, or a film-forming surfactant such as a phospholipid, e.g. as described in WO-A-9211873, WO-A-9217212, WO-A-9222247, WO-A-9409829, WO-A-9428780, WO-A-9503835 or WO-A-9729783). Contrast agent formulations comprising free microbubbles of selected gases, e.g. as described in WO-A-9305819, or comprising a liquid-in-liquid emulsion in which the boiling point of the dispersed phase is below the body temperature of the subject to be imaged, e.g. as described in WO-A-9416739, may also be used.

Other useful gas-containing contrast agent formulations include gas-containing solid systems, for example microparticles (especially aggregates of microparticles) having gas contained therewithin or otherwise associated therewith (for example being adsorbed on the surface thereof and/or contained within voids, cavities or pores therein, e.g. as described in EP-A-0122624, EP-A-0123235, EP-A-0365467, WO-A-9221382, WO-A-9300930, WO-A-9313802, WO-A-9313808 or WO-A-9313809). It will be appreciated that the echogenicity of such microparticulate contrast agents may derive directly from the contained/associated gas and/or from gas (e.g. microbubbles) liberated from the solid material (e.g. upon dissolution of the microparticulate structure). The invention may also be useful in conjunction with contrast agent systems based on microspheres comprising a therapeutic compound as described in e.g. WO98/51284 and WO99/27981.

The disclosures of all of the above-described documents relating to gas-containing contrast agent formulations are incorporated herein by reference.

Gas microbubbles and other gas-containing materials such as microparticles preferably have an initial average size not exceeding 10 μm (e.g. of 7 μm or less) in order to permit their free passage through the pulmonary system following administration, e.g. by intravenous injection. However, larger microbubbles may be employed where, for example, these contain a mixture of one or more relatively blood-soluble or otherwise diffusible gases such as air, oxygen, nitrogen or carbon dioxide with one or more substantially insoluble and non-diffusible gases such as perfluorocarbons. Outward diffusion of the soluble/diffusible gas content following administration will cause such microbubbles rapidly to shrink to a size which will be determined by the amount of insoluble/non-diffusible gas present and which may be selected to permit passage of the resulting microbubbles through the lung capillaries of the pulmonary system.

Where phospholipid-containing contrast agent formulations are employed in accordance with the invention, e.g. in the form of phospholipid-stabilised gas microbubbles, representative examples of useful phospholipids include lecithins (i.e. phosphatidylcholines), for example natural lecithins such as egg yolk lecithin or soya bean lecithin, semisynthetic (e.g. partially or fully hydrogenated) lecithins and synthetic lecithins such as dimyristoylphosphatidylcholine, dipalmitoylphosphatidylcholine or distearoylphosphatidylcholine; phosphatidic acids; phosphatidylethanolamines; phosphatidylserines; phosphatidylglycerols; phosphatidylinositols; cardiolipins; sphingomyelins; fluorinated analogues of any of the foregoing; mixtures of any of the foregoing and mixtures with other lipids such as cholesterol. The use of phospholipids predominantly (e.g. at least 75%) comprising molecules individually bearing net overall charge, e.g. negative charge, for example as in naturally occurring (e.g. soya bean or egg yolk derived), semisynthetic (e.g. partially or fully hydrogenated) and synthetic phosphatidylserines, phosphatidylglycerols, phosphatidylinositols, phosphatidic acids and/or cardiolipins, for example as described in WO-A-9729783, may be particularly advantageous.

Representative examples of materials useful in gas-containing contrast agent microparticles include carbohydrates (for example hexoses such as glucose, fructose or galactose; disaccharides such as sucrose, lactose or maltose; pentoses such as arabinose, xylose or ribose; α-, β- and γ-cyclodextrins; polysaccharides such as starch, hydroxyethyl starch, amylose, amylopectin, glycogen, inulin, pulullan, dextran, carboxymethyl dextran, dextran phosphate, ketodextran, aminoethyldextran, alginates, chitin, chitosan, hyaluronic acid or heparin; and sugar alcohols, including alditols such as mannitol or sorbitol), inorganic salts (e.g. sodium chloride), organic salts (e.g. sodium citrate, sodium acetate or sodium tartrate), X-ray contrast agents (e.g. any of the commercially available carboxylic acid and non-ionic amide contrast agents typically containing at least one 2,4,6-triiodophenyl group having substituents such as carboxyl, carbamoyl, N-alkylcarbamoyl, N-hydroxyalkylcarbamoyl, acylamino, N-alkylacylamino or acylaminomethyl at the 3- and/or 5-positions, as in metrizoic acid, diatrizoic acid, iothalamic acid, ioxaglic acid, iohexol, iopentol, iopamidol, iodixanol, iopromide, metrizamide, iodipamide, meglumine iodipamide, meglumine acetrizoate and meglumine diatrizoate), polypeptides and proteins (e.g. gelatin or albumin such as human serum albumin), and mixtures of any of the foregoing.

The technique described herein would be suitable for use in the infusion of ultrasound products sold under the Trade Names Levovist, Albunex, Optison, Definity, Tmagent, Sonovue, Echogen, Sonogen and Sonazoid.

FIG. 1 depicts a device for inducing a thermal gradient across the body of a syringe. Syringe (1) is inserted into the clamp (6) by sliding between jaws (2, 3) by a motion parallel to the syringe cylindrical axis. The jaws (2, 3) are machined to close tolerances to ensure excellent contact with the syringe body. Jaw (2) is precooled and jaw (3) preheated electrically by Peltier element (4) to induce a temperature gradient across the syringe body. The hot side of the assembly is cooled by the cooling fins (5) in the ambient air.

The principles as described herein can obviously be applied to any disperse system where a difference in density leads to segregation (i.e. flotation or sedimentation) over time, such as emulsions, solid particle dispersions and other disperse systems. Preferably, with floating particles the outlet should be near the highest point of the reservoir in order to maintain homogeneity inside the reservoir. Similarly, with sedimenting particles the outlet should preferably be near the low point of the reservoir.

The technique would also be suitable for simply “stirring” a gas-containing contrast agent formulation and this forms a further aspect of the invention. Hence, viewed from another aspect the invention provides a method of stirring a gas-containing contrast agent formulation in a cylindrical reservoir, e.g. a syringe comprising subjecting said reservoir to a thermal gradient across the body thereof.

The following non-limitative examples serve to illustrate the invention.

Preparation 1—Hydrogenated phosphatidylserine-encapsulated perfluorobutane microbubbles

Hydrogenated phosphatidylserine (5 mg/ml in a 1% w/w solution of propylene glycol in purified water) and perfluorobutane gas were homogenised in-line at 7800 rpm and ca. 40ΕC. to yield a creamy-white microbubble dispersion. The dispersion was fractionated to substantially remove undersized microbubbles (□2 μm) and the volume of the dispersion was adjusted to the desired microbubble concentration. Sucrose was then added to a concentration of 92 mg/ml. 2 ml portions of the resulting dispersion were filled into 10 ml flat-bottomed vials specially designed for lyophilisation, and the contents were lyophilised to give a white porous cake. The lyophilisation chamber was then filled with perfluorobutane and the vials were sealed. Prior to use, normally 2 ml water was added to a vial with lyophilised product and the contents were hand-shaken for several seconds to give a perfluorobutane microbubble dispersion with a concentration range of 500-2000 mill. microbubbles/ml (7-13 μl/ml).

### EXAMPLE 1

Two aluminium blocks each 20×40×65 mm were machined by making a semi-cylindrical groove along their largest surface. The groove was designed to match the outer curvature of a 10 ml BD-Plastipak syringe. One of the blocks was cooled to 5 C. by immersion in cold tap water, while the other block was heated to about 45 C. in warm tap water. A syringe was filled with 8 ml of preparation 1 diluted to approximately 0.3% gas volume and the aluminium blocks were applied to each side of the syringe, keeping the syringe cylindrical axis horizontal. The system was then left undisturbed for 3 minutes, and the contents of the syringe were inspected.

Only minor signs of flotation were seen as small collections of gas bubbles in the upper corners close to the rubber piston and close to the outlet. This was as expected, since the fluid flow pattern will be less vigorous towards the ends of the syringe due to hydrodynamic friction against the inner surfaces.

### EXAMPLE 2

A control experiment was performed with the same syringe and with the same contents as in Example 1 above. The syringe was stored horizontally for 3 minutes without applying the thermal gradient. Obvious flotation was seen as a broad white streak along the upper surface of the syringe and a layer of clear fluid along the bottom of the syringe became evident.

### EXAMPLE 3

A Peltier-element combined cooling and heating device as shown in FIG. 1 is applied to a 10 ml plastic syringe, and is energised from a DC voltage supply for a few minutes until a stable temperature difference is established. The syringe is then filled with 10 ml of the particle dispersion described in Preparation 1.

The syringe is placed horizontally in an IVAC P2000 infusion pump, and the flow rate is set to 1 ml/min. Samples of the fluid leaving the syringe are taken at regular intervals and particle concentrations and size distributions are determined by Coulter counter. The analytical results are found to be essentially unchanged during the 10 minutes of emptying the syringe.

### EXAMPLE 4

The experiment described in example 3 is repeated, however without applying the cooling and heating device. The analytical results now show that the particle concentration and size distribution vary considerable during the 10 minutes of emptying the syringe.

1. A method of administering a gravity segregating dispersion to a subject by continuous infusion, wherein said dispersion is controllably delivered from a reservoir exposed to a thermal gradient across the body thereof.

2. A method as claimed in claim 1 wherein the reservoir comprises a syringe.

3. A method as claimed in claim 1 or 2 wherein the dispersion comprises a contrast agent.

4. A method as claimed in any of the claims 1 to 3 wherein the reservoir is in contact with one or several thermal sources.

5. A method as claimed in any of the claims 1 to 4 wherein the reservoir is in contact with 2 thermal sources of different temperatures.

6. A method as claimed in any of the claims 1 to 5 wherein the reservoir is exposed to temperatures not greater than 45° C. or less than 5° C.

7. A method as claimed in any of the claims 1 to 6 wherein the reservoir is held between two jaws of different temperatures.

8. A method as claimed in any of the claims 1 to 7 wherein a Peltier element is used to achieve the thermal gradient.

9. A method as claimed in any of the claims 1 to 7 wherein the dispersion is controllably delivered from the reservoir and thereafter is admixed with a flushing medium prior to administration to the subject.

10. An apparatus useful in the administration of a gravity segregating dispersion, by continuous infusion, said apparatus comprising (i) a reservoir adapted to retain a gravity segregating dispersion; (ii) means to induce a thermal gradient across the body of the reservoir; (iii) optionally means to allow co-administration of said gravity segregating dispersion with a flushing medium and (iv) conduit means for conducting said dispersion and optionally said flushing medium to an injection device.

11. An apparatus as claimed in claim 10 wherein the reservoir comprises syringe driver means.

12. An apparatus as claimed in claim 10 or 11 wherein the means to induce a thermal gradient is adapted to expose the reservoir to temperatures not greater than 45° C. or less than 5° C.

13. An apparatus as claimed in any of claims 9 to 12 wherein the means to induce a thermal gradient comprises at least one thermal source contacting not more than half of the reservoir body.

14. Use of an apparatus as claimed in any of claims 9 to 13 in administration of a gravity segregating dispersion to a subject by continuous infusion.
